(19) United States
(12) Patent Application Publication
(10) Pub. No.: US 2018/0366214 A1
(43) Pub. Date: Dec. 20, 2018

(54) ORGANIZING APPARATUS AND METHODS OF SAMPLE MEDICATIONS TO GUIDE PERIODIC REFILLING OF MEDICINE TRAYS

(71) Applicant: Susan Dzialo, Glastonbury, CT (US)

(72) Inventor: Susan Dzialo, Glastonbury, CT (US)

(21) Appl. No.: 16/008,091

(22) Filed: Jun. 14, 2018

Related U.S. Application Data
(60) Provisional application No. 62/519,368, filed on Jun. 14, 2017.

(51) Int. Cl. G16H 20/10; G06Q 10/08

(52) U.S. Cl. CPC G16H 20/10; G06Q 10/087

(57) ABSTRACT

Methods and systems for managing the organization of medications for the periodic filling of medicine trays are disclosed. The methods and systems provide a sample physical specimen for multiple medications to enable a patient or a caregiver to periodically fill a pill tray using the physical specimens quickly and accurately.

### CROSS REFERENCE TO RELATED APPLICATIONS

This application claims the benefit of U.S. Provisional Patent Application Ser. No. 62/519,368 filed 14 Jun. 2017. The disclosure of the application above is incorporated herein by reference in its entirety.

### BACKGROUND OF THE INVENTION

### Field of the Art

Embodiments of the disclosure generally relate to medical devices and more particularly to “out-patient” management of medication, where a patient or care-giver must arrange medication, vitamins, and/or supplements for an on-going regimen.

### Description of the Related Art

It is common knowledge that people today take many medications, supplements and vitamins on a daily basis. Many of these substances have to be taken on a schedule and with certain special instructions. Unless the person is hospitalized the person, the person's family or a caregiver must manage the medications. Some medications may need to be taken multiple times a day—morning, noon, evening, and bedtime—and some weekly or monthly. Many medications look similar and have multiple names (brand and generic) and have names that are difficult for the lay person to recognize.

There exists many medicine trays and pill organizers for the patient. A typical prior art pill organizer is a tray having multiple wells divided into compartments that correspond to times of day and days in a week. The patient puts the appropriate medications into the appropriate well, usually at the beginning of a week, and then dispenses the medication at the appropriate time. A missed dose of medication is visually evident by inspection of a well whose dosing time has lapsed. This arrangement is often accomplished by pre-filling a medicine tray with pills, usually for a seven-day supply. The medicine tray often further organizes the daily doses of pills for prescribed dosing time or time-of-day ingestion: morning, noon, evening, bedtime for example.

Prior art medicine trays, such as tray 30 in FIG. 3, are helpful to a patient but they do have short comings when used on their own. Correctly filling multiple medications and supplements, each with its own frequency, into an array, for example, of (28) wells in the medicine tray (7 days×4 time-of-day options) can become confusing and lead to errors. In many cases the patient is required to organize the medication by themselves from a list provided by a medical practitioner or pharmacist. The patient must match the name of a particular medication on the list with a label on a medicine packaging (pill bottle in many cases) in order to properly fill the wells of a pill tray. This is often a long, complicated and frustrating exercise that must be repeated on a frequent basis.

### SUMMARY OF THE DISCLOSURE

In some aspects of the present disclosure, a medicine management apparatus is disclosed that includes a backing board having a plurality of pocket assemblies positioned thereon and arranged as a row and the plurality of pocket assemblies having a plurality of pockets adapted to receive a plurality of medications. The medicine management apparatus further includes a plurality of data fields positioned on the backing board and corresponding to the plurality of pocket assemblies and a plurality of data fields positioned as a column on the backing board and corresponding to the plurality medications.

In other aspects of the present disclosure a method of organizing medications is disclosed that includes obtaining information about multiple medications and obtaining a sample medication for each of the multiple medications. Arranging multiple pockets in rows on a backing board wherein each row corresponds to a dosing time. The method further provides for entering the information for each of the multiple sample medications on the backing board positioned as plurality of columns and dispensing one of the multiple sample medications into each of the pockets in one of the columns corresponding to the information for one of the multiple rows.

The methods also include obtaining a pill organizer having multiple receptacles arranged thereon in accordance with the dosing time and one of the days a week and dispensing a dosing medication matching the sample medication in each of the pockets in one of the rows into one of the receptacles in the pill organizer corresponding to the dosing time for each of the days of the week.

### BRIEF DESCRIPTION OF THE DRAWINGS

So that the manner in which the above recited features of the present disclosure can be understood in detail, a more particular description of the invention, briefly summarized above, can be had by reference to embodiments, some of which are illustrated in the appended drawings. It is to be noted, however, that the appended drawings illustrate only typical embodiments of this invention and are therefore not to be considered limiting of its scope, for the invention can admit to other equally effective embodiments.

FIG. 1 is a graphical representation of a medication management device in accordance with certain aspects of the present disclosure.

FIG. 2 is a graphical representation of a prior art medication information form.

FIG. 3 is a graphical representation of a prior art pill tray.

### DETAILED DESCRIPTION

The present disclosure describes an “out-patient” management of medication, where a patient or care-giver must arrange medication, vitamins, and/or supplements for an on-going regimen. For purposes of clarity and brevity, the terms medication, vitamins, supplements and the like may be used interchangeably herein, and may commonly be referred to herein as a pill, to describe a substance that is orally ingested by a person on a regular or semiregular basis.

The medication management device and method of the present disclosure addresses the short comings in the prior art by means of a sample physical specimen of the patient's actual pills, reflecting the periodic medication regimen along with other information related to the pill as will be more fully described herein below. The medication management device and method of the present disclosure is intended to be used as an updateable guide, or compliance device, each time a patient's medicine tray is re-filled.

Once the dispensing of a selected medication is reviewed in the sample physical specimen, that particular medication can be quickly and accurately distributed from a supply of such medication, a bottle for example, into the appropriate time-of-day wells for each day in the medicine tray, completing that particular medication's distribution for the week or selected period. The process is repeated for each medication according to the sample physical specimen.

In accordance with the present disclosure, and as will be more fully described herein after, the physical display of a sample physical specimen addresses at least two potential sources of confusion that exist in the prior art. One such source of confusion is caused by changes to prescription instructions such as adding or eliminating medications, or changing dosage and/or timing, as medical practitioners adjust medications. The other source of confusion is caused by changes to the physical appearance of medication as prepared by pharmaceutical manufacturers at the time prescriptions are re-filled. Changes to size, shape, and color commonly occur, especially as pharmacies change providers for better pricing, or generic products become available.

The medication management device and method of the present disclosure accommodates easy and timely replacement of the pill samples. This will allow for adding or eliminating medications, or adjusting dosage patterns as soon as prescribed. Pill samples can immediately be replaced if their manufacture changes at time of re-fill from the pharmacy, so the pill guide remains visually accurate.

### Examples of Organizing Apparatus of Sample Pills

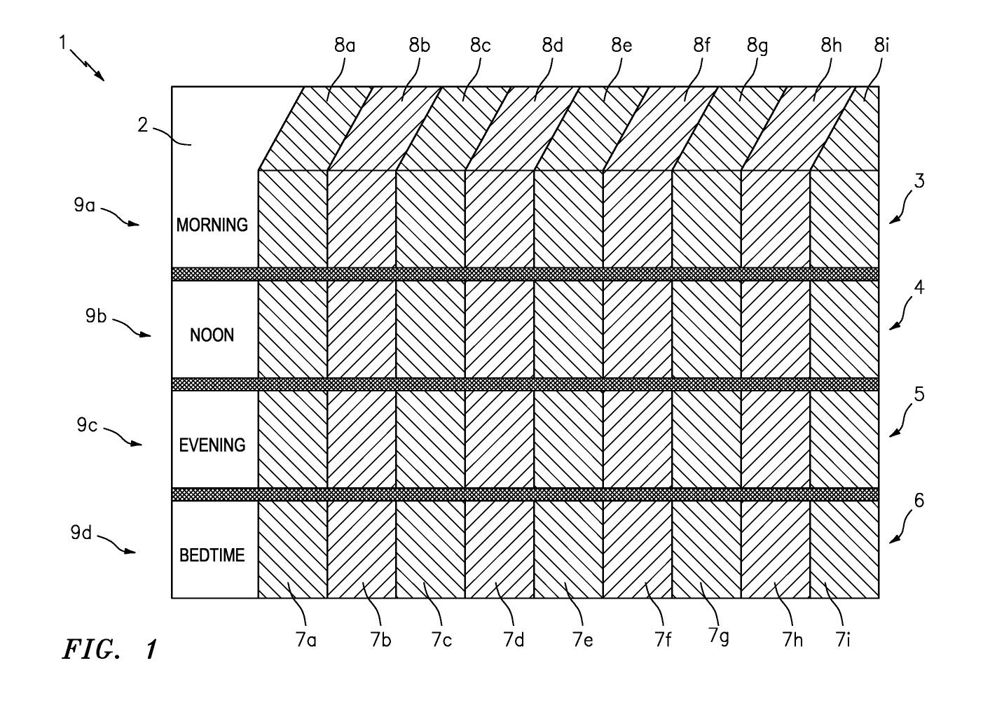

With reference to FIG. 1, there is shown an embodiment of a medication management device 1 of the present disclosure. In this particular embodiment medication management device 1 is comprised of a backing board 2 which backing board may be comprised of any suitable rigid material such as cardboard or plastic. Positioned on backing board 2 are four sets of sample physical specimen assemblies 3, 4, 5 and 6. Sample physical specimen assemblies 3, 4, 5 and 6 are comprised of pockets 7a-7i wherein the pockets are comprised of a transparent material and are arranged in rows and are adapted to receive a particular medication within the pocket and to provide a visual indication of the medication positioned therein. In this particular embodiment, each pocket 7a-7i is approximately 1.25″ by 1″ with a pocket opening of 1″. It should be apparent to those skilled in the art that sample physical specimen assemblies 3, 4, 5 and 6 are arranged to accommodate a total of 36 individual sample physical specimens. Positioned along the top portion of backing board 2 are data field areas 8a-8i wherein a user (not shown) can write information pertaining to the particular medication provided in the pockets in the column 7a-7i directly beneath each data field. Also included on backing board 2 are data fields 9a-9d arranged to correspond with a row of pockets and in this particular embodiment indicate the period of the day or dosing time, namely morning, noon, evening, and bedtime.

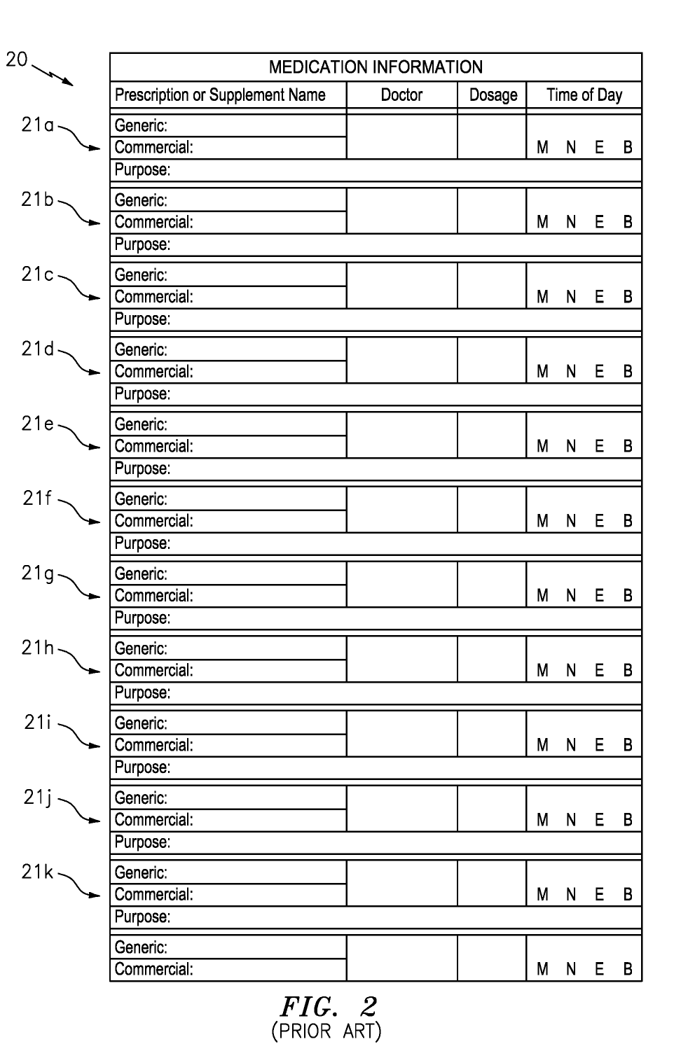

It is contemplated by the present disclosure that medication management device 1 can part be of an overall package (not shown) intended to support a patient's routine in maintaining the periodic dispensing of a conventional medicine tray. For example, such a package can include a carrying device such as a bag, a prior art medicine tray, a list of medications form, such as medication information form 20 as shown in FIG. 2, for capturing additional information about each medication. Medication information form 20 can be carried to healthcare and pharmacy visits, and revised there as appropriate. This form can be a bridge between medication instructions and updates to medication management device 1, keeping it current, as will be more fully described herein after. For instance, when the prescribing doctor provides updated information concerning a new medication or dosage an updated sample can be added to the form and can be added to the organizer as described herein below. In addition, updated information from a prescribing doctor can delete a medication and the information can be removed and the obsolete sample medication can be removed from the organizer. In addition, such a package can also include a pencil with an eraser, or other rewritable device, for easily revising updated information on the medication information form 20, and writing data fields 8a-8i of medication management device 1 providing updated column information, thereby extending the useful life of the medication management device with rewritable text. Tweezers may also be included to manage pills which are difficult to grasp. The carrying device can be comprised of any material suitable to hold the aforementioned items and can hold the patient's current supply of active medication. In addition, an instruction guide for completing the sample physical specimen as well as general suggestions could also be included.

Now referring to FIG. 2, there is shown an example of medical information form 20 for use with the present disclosure. A user populates the fields 21a-21k with each particular medication indicating the name of the medication, a summary of its purpose, the name of the prescribing doctor (if any), the prescribed dosage level and dosage timing. It should be apparent to those skilled in the art that any type form containing sufficient information to fulfill the procedure outlined herein below is contemplated within the present disclosure.

Referring to FIGS. 1 and 2, in operation, a user starts with an up-to-date listing of medications such as may be provided on medication information form 20 and a supply of the medications listed thereon. The user transfers the relevant information from the first medication listed in data field 21a of medication information form 20 to data field 8a on medication management device 1. The user then populates the appropriate pocket(s) with the medication listed in data field 21a, including the correct dosage level and timing. For example, if the dosage level of the medication listed in data field 21a indicates that two pills are required to be taken at morning, noon and night, then the user takes the medication from the patient's supply and inserts two pills into pockets 7a in each of pocket assemblies 3, 4 and 5. The user then completes medication management device 1 by repeating the above outlined procedure for the remainder of medications listed in data fields 21b-21k on medication information form 20.

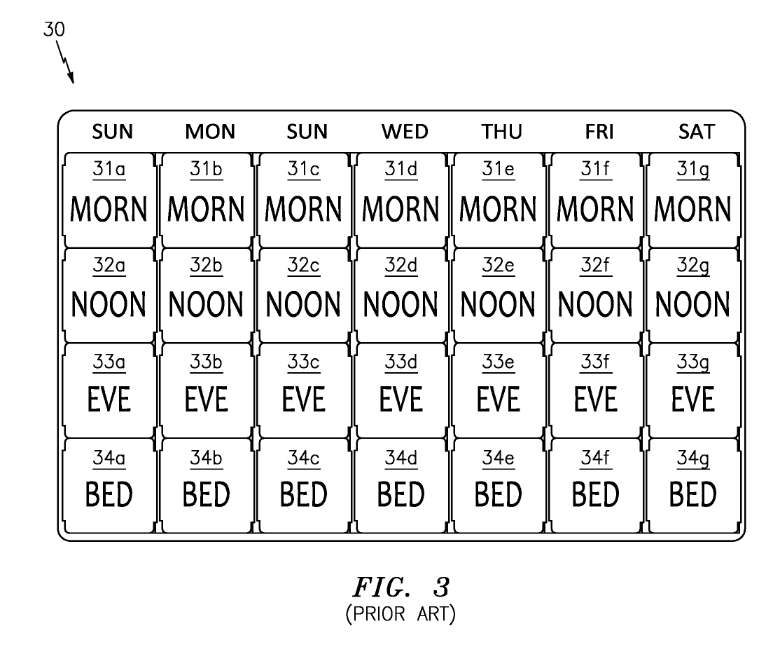

Now with reference to FIGS. 1 and 3, the user then, using the completed medication management device 1 as a guide, fills a prior art pill tray, such as pill tray 30 comprised of a plurality of wells (or receptacles) having lids that open for dispensing medications and close for enclosing medications therein. Starting with the dosing time “MORNING” indicated in data field 9a of medication management device 1, and using both the visual information from the sample physical specimen and the information in data fields 8a-8i, a user replicates all of the medications contained in pockets 7a-7i in pocket assembly 3 into the well 31a marked “SUN” “MORN” in pill tray 30. The user then repeats this procedure for wells 31b-31g. Next, using the dosing time “NOON” indicated in data field 9b of medication management device 1, and using both the visual information from the sample physical specimen and the information in data fields 8a-8i, a user replicates all of the medications contained in pockets 7a-7i in pocket assembly 4 into the well 32a marked “SUN” “NOON” in pill tray 30. The user then repeats this procedure for wells 32b-32g. Next, using the dosing time “EVENING” indicated in data field 9c of medication management device 1, and using both the visual information from the sample physical specimen and the information in data fields 8a-8i, a user replicates all of the medications contained in pockets 7a-7i in pocket assembly 5 into the well 33a marked “SUN” “EVE” in pill tray 30. The user then repeats this procedure for wells 33b-33g. Next, using the dosing time “BEDTIME” indicated in data field 9d of medication management device 1, and using both the visual information from the sample physical specimen and the information in data fields 8a-8i, a user replicates all of the medications contained in pockets 7a-7i in pocket assembly 6 into the well 34a marked “SUN” “BED” in pill tray 30. The user then repeats this procedure for wells 34b-34g. This procedure can be repeated on a weekly basis.

While the foregoing is directed to only certain embodiments of the present disclosure certain observations of the breadth of the present disclosure should be made. For instance, although medication management device 1 is as shown and described, the present disclosure includes other types of devices that could hold and display sample physical specimens of medication such as devices having wells.

While the foregoing is directed to embodiments of the present disclosure, other and further embodiments of the invention can be devised without departing from the basic scope thereof, and the scope thereof is determined by the claims that follow.

What is claimed is:

1. A medicine management apparatus comprising:
a backing board;
a plurality of pocket assemblies positioned in a plurality of rows on the backing board, the plurality of pocket assemblies having a plurality of pockets adapted to receive a plurality of medications;
a plurality of first data fields positioned on the backing board and corresponding to the plurality of pocket assemblies; and
a plurality of second data fields positioned as a plurality of columns on the backing board and corresponding to the plurality of medications.

2. The apparatus of claim 1 wherein each of the plurality of first data fields indicate a dosing time.

3. The apparatus of claim 1 wherein each of the plurality of second data fields indicate information about one of the plurality of medications.

4. The apparatus of claim 1 wherein the plurality of second data fields is adapted to receive rewritable text.

5. The apparatus of claim 1 wherein the backing board is comprised of a rigid material.

6. The apparatus of claim 1 wherein at least a portion of the plurality of pockets are comprised of a transparent material.

7. The apparatus of claim 1 wherein each of the plurality of pockets include an opening to receive the plurality of medications therethrough.

8. The apparatus of claim 1 wherein the plurality of pockets are comprised of a well and wherein the well includes a lid having an open position and a closed position and wherein the plurality of medications are received into the well when the lid is in the open position.

9. A method of organizing medications comprising:
obtaining information about each of a plurality of medications;
obtaining a plurality of sample medications for each of the plurality of medications;
providing a backing board having a plurality of pockets positioned in a plurality of rows thereon;
indicating a dosing time corresponding to each of the plurality of rows; and
entering the information for each of the plurality of sample medications on the backing board positioned as plurality of columns; and
dispensing one of the plurality of sample medications into each of the pockets in one of the plurality of columns corresponding to the information for the one of the plurality of rows.

10. The method of claim 9 further comprising:
obtaining a pill organizer having a plurality of receptacles arranged thereon in accordance with the dosing time and one of the days a week; and
dispensing a dosing medication matching the sample medication in each of the pockets in one of the plurality of rows into one of the plurality of receptacles in the pill organizer corresponding to the dosing time for each of the days of the week.

11. The method of claim 9 wherein the information comprises any of a name of the plurality of medications, a dosage, a purpose, a name of prescribing doctor and the dosing time.

12. The method of claim 9 further comprising:
obtaining updated information about an updated medication;
obtaining an updated sample medication for the updated medication;
entering the updated information in an updated column;
dispensing the updated sample medication in to at least one pocket in the updated column.

13. The method of claim 12 further comprising removing an obsolete sample medication from at least one of the plurality of pockets.

14. The method of claim 10 further comprising dispensing the plurality of medications in the pill organizer in accordance with the dosing time and a day of the week.

15. The method of claim 14 further comprising redispensing a dosing medication matching the sample medication in each of the pockets in one of the plurality of rows into one of the plurality of receptacles in the pill organizer corresponding to the dosing time for each of the days of the week.
